(19) United States
(12) Patent Application Publication
(10) Pub. No.: US 2021/0100539 A1
(43) Pub. Date: Apr. 8, 2021

(54) ENDOCYTE CANNULA

(71) Applicant: All Cape Gynecology LLC, Hyannis, MA (US)

(72) Inventor: Lucia Cagnes, Sandwich, MA (US)

(21) Appl. No.: 17/126,660

(22) Filed: Dec. 18, 2020

Related U.S. Application Data
(63) Continuation-in-part of application No. 15/117,994, filed on Aug. 10, 2016, now abandoned.
(60) Provisional application No. 61/938,377, filed on Feb. 11, 2014.

(51) Int. Cl. A61B 10/02

(52) U.S. Cl. CPC A61B 10/0291; A61B 2010/009; A61B 10/0283

(57) ABSTRACT

An endocyte cannula is designed to provide improved and more productive Pap smear tests. The endocyte cannula includes a proximal portion and a distal portion. The proximal portion may include a fitting to connect to a user-actuated tool, such as a syringe. The distal portion may include a corkscrew feature or a lateral wing feature to facilitate movement of the cannula through dense tissue as well as provide scraping or brushing capabilities. The shaped scraping or brushing features may be provided on an attachment that is attachable to a shaft of the endocyte cannula. The attachments may allow customization of the endocyte cannula based on the physiology and anatomy of a patient. The distal portion may further include a plurality of apertures to aspirate mucus and reduce the risk of trauma or perforation. The apertures may extend outside of the attachment or shaped scraping or brushing features, and the distal portion may be narrower than the shaped scraping or brushing features to facilitate access into the endocervical canal and aspiration and collection of a cell sample.

### CROSS-REFERENCE TO RELATED APPLICATIONS

This application is a continuation-in-part of U.S. application Ser. No. 15/117,994 entitled “ENDOCYTE CANNULA” filed on Aug. 10, 2016, which is a 371 national stage filing of and claims priority to PCT Application No. PCT/US15/15449 entitled “ENDOCYTE CANNULA” filed on Feb. 11, 2015, which claims the benefit of U.S. Provisional Application No. 61/938,377 entitled “ENDOCYTE CANNULA,” filed on Feb. 11, 2014, each of which is hereby incorporated by reference in its entirety.

### FIELD OF INVENTION

The invention pertains to an endocyte cannula for use in Pap smears and other medical related tests.

### BACKGROUND OF THE INVENTION

Papanicolaou tests (“Pap tests” or Pap smears) are commonly performed tests that have been proven to be highly valuable in the early detection of cervical pre-cancerous and cancerous growths. The Pap test refers to the collection of cells from the cervical face, the endocervical canal, and occasionally from the vaginal wall. The collected cells are subsequently “smeared” onto a microscope plate or deposited and mixed into a broth and analyzed for evidence of pre-cancerous or cancerous growth. A periodic Pap test permits the early detection of malignant cells, which enables the treatment of cervical pre-cancerous and cancerous growths.

Numerous devices have been developed to assist in the collection of cells during a Pap test. One such device is a wooden or plastic spatula. Such spatulas are inexpensive and can be effective at collecting cells from the cervical face. However, spatulas have proven to be less than effective in collecting adequate cell samples from the endocervical canal. This is a potentially serious short-coming, because any sample that does not include endocervical cells is deemed to be an inadequate Pap smear sample. That is to say, the proper interpretation and diagnosis of the state of the cells is inconclusive unless a sufficient number of cells are collected from the endocervical canal.

Other devices that are useful in collecting cells during Pap tests include certain bristle brushes in conjunction with a spatula. In this regard, the bristle brushes are capable of obtaining endocervical cells during sampling, however bristle brushes are not always able to penetrate a narrow endocervical canal that may be completely closed in a given patient.

Pap tests have proven to be useful in the early detection of malignant cells and are related to a reduction in the incidence and death rate due to cervical cancers. Improvements to sampling devices useful in collecting cells during Pap tests will be welcomed by the medical community and patients alike. Therefore, there is a need for an improved sampling device that is able to overcome at least one of the deficiencies noted above regarding other sampling devices.

### SUMMARY OF THE INVENTION

An endocyte cannula is generally provided. A hybrid endocyte cannula is generally provided. The endocyte cannula is designed to provide improved and more productive Pap smear tests. The endocyte cannula is designed to provide an improved quality and quantity of cervical mucous needed for a Pap smear test. The endocyte cannula includes a proximal portion and a distal portion. The proximal portion may include a fitting to connect to a user-actuated tool, such as a syringe. The distal portion may include a corkscrew feature to facilitate movement of the cannula through dense tissue. The distal portion may further include a plurality of holes to aspirate mucus and reduce the risk of trauma or perforation to the patient.

The distal portion may include a suction element and a mechanical brushing or scraping element. The suction element may be provided by a bulb. The mechanical brushing or scraping element may be provided by one or more lateral wings. The suction element may be able to retrieve cell samples from the endocervical canal and the cervix. The mechanical brushing or scraping element may collect cells from the endocervical canal and the cervix. The width, shape, and orientation of the mechanical brushing or scraping element may be customized by interchangeable tip heads that are attachable to the distal portion. The customization of the mechanical brushing or scraping element may vary based on the user and the depth or shape of the endocervical canal of a patient.

In one embodiment, the present technology includes a medical device comprising a tubular body having a proximal end and a distal end, wherein the tubular body is rigid and includes a hollow channel, wherein the distal end is configured to receive a scraping attachment, and wherein the proximal end includes a user-control mechanism. In one aspect, the scraping attachment is triangular. In one aspect, the scraping attachment includes two lateral wings. In one aspect, the scraping attachment is corkscrew shaped. In one aspect, the distal end further comprises at least one aperture, the aperture having a first opening through an outer wall of the distal end and a second opening through an inner wall of the hollow channel of the tubular body. In one aspect, the scraping attachment includes at least one aperture, the aperture having a first opening through an outer wall of the scraping attachment and a second opening through an inner wall a hollow channel of the scraping attachment, wherein the hollow channel of the scraping attachment is fluidly connected to the hollow channel of the tubular body when the scraping attachment is attached to the tubular body.

In one aspect, the scraping attachment includes a receiving passage configured to receive the distal end of the tubular body there through. In one aspect, the receiving passage extends through an entire length of the scraping attachment, having a first opening on a first end and a second opening on a second end, and wherein the distal end of the tubular body extends into the first opening, through the length of the scraping attachment, and at least a potion of the distal end of the tubular body extends out of the second opening, when the scraping attachment is attached to the tubular body. In one aspect, at least one aperture is located on the portion of the distal end that extends through the scraping attachment and second opening when the scraping attachment is attached to the tubular body.

In one aspect, the user-control mechanism is a syringe. In one aspect, the user-control mechanism is a bulbous end. In one aspect, the user-control mechanism interfaces with the hollow tubular body to create a force through the medical device. In one aspect, the medical device is configured to aspirate bodily tissues. In one aspect, the distal end and scraping attachment are configured to be received into a body cavity. In one aspect, the scraping attachment is configured to collect cells from the body cavity. In one aspect, the body cavity is an endocervical canal. In one aspect, the hollow tubular body comprises steel, stainless steel, plastic, rubber, or a combination of two or more thereof.

In one embodiment, the present technology includes a method for completing a Papanicolaou test comprising: inserting a medical device into an endocervical canal, the medical device comprising a hollow tubular body having a proximal end and a distal end, wherein the proximal end connects to a user-control mechanism and the distal end comprises a scraping component and at least one aperture; and collecting cells from the endocervical canal with the medical device. In one aspect, the distal end and scraping component advance through bodily tissue. In one aspect, the at least one aperture aspirates bodily tissues.

In one embodiment, the present technology includes a medical device having a hollow tubular body having a proximal end and a distal end, wherein the proximal end is configured to connect to a user-control mechanism. In one aspect, the distal end of the hollow tubular body includes a corkscrew-shaped portion having at least one spiral. In one aspect, the distal end of the hollow tubular body includes an attachable mechanical brushing or scraping element. In one aspect, the distal end includes at least one aperture, the aperture having a first opening through an outer wall of the medical device and a second opening through the hollow tubular body. In one aspect, the at least one aperture is configured to aspirate bodily tissues. In one aspect, the proximal end includes a fitting configured to connect to the user-control mechanism. In one aspect, the fitting is a luer lock. In one aspect, the user-control mechanism is a syringe. In one aspect, the syringe interfaces with the hollow tubular body to create a force through the medical device. In one aspect, the hollow tubular body is configured to be received into a body cavity. In one aspect, the hollow tubular body is configured to collect cells from a body cavity. In one aspect, the body cavity is an endocervical canal. In one aspect, the hollow tubular body comprises steel, stainless steel, plastic, rubber, or a combination of two or more thereof.

In one embodiment, the present technology provides a method for completing a Papanicolaou tests includes inserting a medical device into an endocervical canal. The medical device includes a hollow tubular body having a proximal end and a distal end, wherein the proximal end connects to a user-control mechanism and the distal end includes a shape to acquire cells, such as a corkscrew-shaped portion comprising at least one spiral and at least one aperture or a wing-shaped attachment positionable onto a shaft of the medical device, the shaft comprising an aperture. Cells are collected from the endocervical canal with the medical device. In one aspect, the corkscrew-shaped portion advances through bodily tissue. In one aspect, the wing-shaped attachment advances through bodily tissue. In one aspect, the apertures aspirate bodily tissues.

In one embodiment, the present technology provides a kit for gathering endocervical canal tissue. The kit includes a medical device having a hollow tubular body with a proximal end and a distal end, wherein the proximal end is configured to connect to a user-control mechanism. The kit also includes a user-control mechanism. In one aspect, the distal end of the medical device further comprises a corkscrew-shaped portion comprising at least one spiral and at least one aperture. In one aspect, the distal end of the medical device further comprises a wing-shaped portion comprising at least one edge and at least one aperture. In one aspect, the user-control mechanism is selected from a manually actuated tool, a robot actuated tool, or an automated tool, or any combination of two or more thereof.

### BRIEF DESCRIPTION OF THE DRAWINGS

The invention may be better understood by reference to the following detailed description taken in connection with the following illustrations, wherein:

FIG. 1 is a plan view of an endocyte cannula.

FIG. 2 is a cross-sectional view of the endocyte cannula of FIG. 1 along the line 2.

FIG. 3 is a perspective view of a different embodiment of an endocyte cannula.

FIG. 4 is a perspective view of the endocyte cannula selectively secured to a syringe.

FIG. 5 is a perspective view of the endocyte cannula selectively secured to a syringe.

FIG. 6 is a perspective view of the fitting of the endocyte cannula.

FIG. 7 is a perspective view of a second embodiment of an endocyte cannula.

FIG. 8 is a plan view of the endocyte cannula.

FIG. 9 is a side view of the endocyte cannula.

FIG. 10 is a top view of the endocyte cannula.

FIG. 11 is an enlarged plan view of a shaft of the endocyte cannula.

FIG. 12 is an enlarged perspective view of an aperture of the endocyte cannula.

FIG. 13 is a perspective view of the scraping attachment of the endocyte cannula.

FIG. 14 is a top view of the scraping attachment of the endocyte cannula.

FIG. 15 is a side view of the scraping attachment of the endocyte cannula.

FIG. 16 is a plan view of the separated shaft and scraping attachment of the endocyte cannula.

FIG. 17 is an enlarged plan view of the separated shaft and scraping attachment of the endocyte cannula.

FIG. 18 is an enlarged perspective view of the separated shaft and scraping attachment of the endocyte cannula.

FIG. 19 is an enlarged perspective view of the attached shaft and scraping attachment of the endocyte cannula.

### DETAILED DESCRIPTION OF THE INVENTION

Reference will now be made in detail to exemplary embodiments of the present invention, examples of which are illustrated in the accompanying drawings. It is to be understood that other embodiments may be utilized and structural and functional changes may be made without departing from the respective scope of the invention. Moreover, features of the various embodiments may be combined or altered without departing from the scope of the invention. As such, the following description is presented by way of illustration only and should not limit in any way the various alternatives and modifications that may be made to the illustrated embodiments and still be within the spirit and scope of the invention.

An endocyte cannula disclosed herein is designed to provide improved and more productive Pap smear tests. The endocyte cannula includes a proximal portion and a distal portion. The proximal portion may include a fitting to connect to a user-actuated tool, such as a syringe. The distal portion may include a corkscrew feature or a lateral wing feature to facilitate movement of the cannula through dense tissue as well as provide scraping or brushing capabilities. The shaped scraping or brushing features may be provided on an attachment that is attachable to a shaft of the endocyte cannula. The attachments may allow customization of the endocyte cannula based on the physiology and anatomy of a patient. The distal portion may further include a plurality of apertures to aspirate mucus and reduce the risk of trauma or perforation. The apertures may extend outside of the attachment or shaped scraping or brushing features, and the distal portion may be narrower than the shaped scraping or brushing features to facilitate access into the endocervical canal and aspiration and collection of a cell sample.

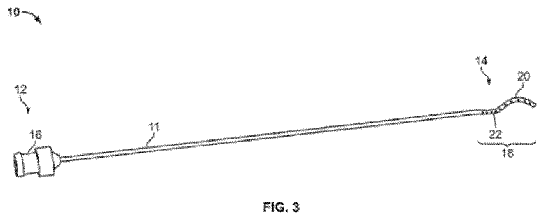

An endocyte cannula 10, 100 is depicted in FIGS. 1, 3, and 8. The endocyte cannula 10, 100 may be configured for use in Pap smear tests and other medical related tests. As described below and illustrated in the Figures, the endocyte cannula 10, 100 may improve the results and method of conducting a Pap smear. While the endocyte cannula 10, 100 is shown and described in use with a Pap smear, the present teachings are not limited to such. The endocyte cannula 10, 100 may be utilized in any appropriate medical related testing.

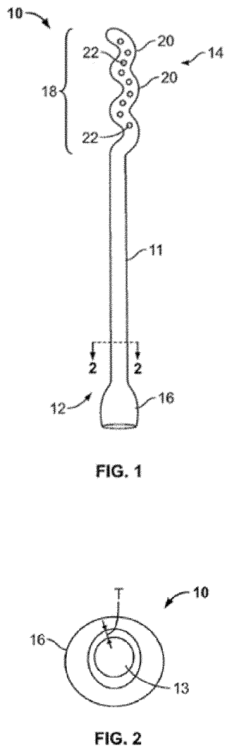

FIG. 1 depicts the endocyte cannula 10. The endocyte cannula 10 may be comprised of any appropriate material, including, without limitation, steel, stainless steel, plastic, rubber, a combination of the foregoing or any other appropriate material. In an embodiment, the endocyte cannula 100 may be comprised of steel, stainless steel, plastic, rubber, a combination of the foregoing, including, without limitation, medical grade plastic and may be disposable or single-use. The endocyte cannula 10 and 100 may be made available in translucent colors. The endocyte cannula 10 may include a body 11. The body 11 may be generally hollow and may include any appropriate wall thickness, such as T in FIG. 2. T may be any appropriate thickness, including for example, 0.1 mm-10 mm. As shown in FIG. 2, the body 11 may include a generally hollow tubular portion 13, which may be used in the aspiration process described in more detail below.

The endocyte cannula 10 may include a proximal end 12 and a distal end 14. The proximal end 12 may be generally configured to connect to a user-control mechanism. By way of a non-limiting example, the user control mechanism may include a syringe 15 or the like. The distal end 14 may be designed to generally increase effectiveness of use and allow for aspiration, as further described below.

The endocyte cannula 10 may be any appropriate size and shape. By way of a non-limiting example, the endocyte cannula 10 may be approximately 12 cm in total length. This length will help the endocyte cannula 10 to effectively maneuver through the cervix and/or the endocervical canal to effectively obtain the desired cells. It will be appreciated, however, that then length of the endocyte cannula 10 may be longer or shorter than 12 cm, and may be any appropriate length. For example, the endocyte cannula 10 may be 5 cm to 50 cm in length. The diameter of the endocyte cannula 10 may be similar to that of a 20 gauge needle, such as between 0.6 mm and 1.0 mm. Alternatively, the diameter of the endocyte cannula 10 may be greater or smaller than that of a 20 gauge needle, for example, the diameter may range from 0.1 mm to 10 mm. The diameter may be consistent throughout the entirety of the endocyte cannula 10 or may vary across the length or at different portions of the endocyte cannula 10.

The proximal end 12 may be configured to connect to a user control mechanism. For example, the proximal end 12 may include a fitting 16, such as a taper or other feature, to interface with the syringe 15. In an embodiment, the fitting 16 may comprise a “luer taper” or “luer lock” or any other appropriate type of fitting or means or connection to provide an interface between the endocyte cannula 10 and the syringe 15 or other tool. While the syringe 15 is shown as being used with the endocyte cannula 10, it should be understood that any appropriate tool may be used and the teachings are not limited to the syringe 15 shown and described.

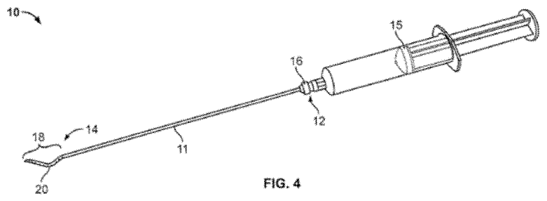

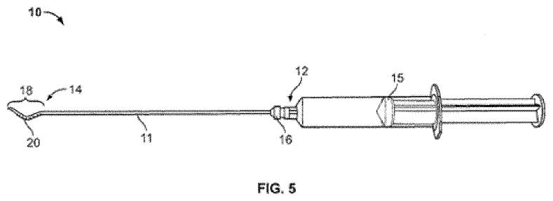

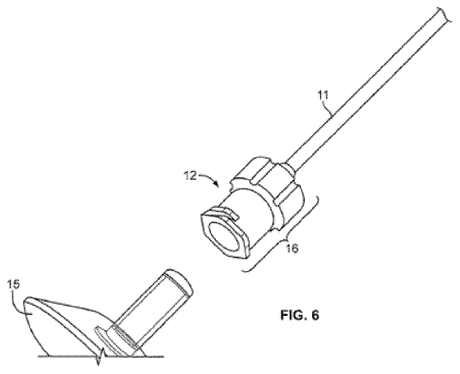

In some embodiments, the syringe 15 may be selectively attached to the fitting 16, as shown in FIGS. 4-6. The syringe 15 may interface with the generally hollow endocyte cannula 10, specifically, the tubular portion 13 to apply a force, pressure or suction therethrough. When suction is applied to the cervix, a representative sample of the cells may be collected into the shaft portion or body of the endocyte cannula 10 and then disposed into the designated jar or slide. While the endocyte cannula 10 is described as connected to the syringe 15, it will be appreciated that the proximal end 12 may be connected to any appropriate tool, including any manually actuated, robot actuated or automated tool.

The distal end 14 may be configured to enhance the functionality and use of the endocyte cannula 10, and/or to receive an attachment that assists in these ends. The distal end 14 may itself include a shape configured to provide mechanical scraping, brushing, or otherwise facilitate the positioning of the device during a Pap smear and the collection and retrieval of a desired cellular specimen, including cervical cells (e.g. from the face of the cervix), endocervical cells (e.g. from the endocervical canal), and/or cervical mucous or the distal end 14 may be configured to receive an attachment, wherein the attachment includes a shape that is similarly configured to provide mechanical scraping, brushing, or otherwise facilitate the positioning of the device during a Pap smear and the collection and retrieval of a desired cellular specimen, including cervical cells (e.g. from the face of the cervix), endocervical cells (e.g. from the endocervical canal), and/or cervical mucous. Although the corkscrew-shape is generally described herein as being part of the distal end 14 and the wing-shape is generally described as being an attachment for positioning onto the distal end 140, it is noted that the reverse is also contemplated herein, where the cork-screw shape is generally provided as an attachment for positioning onto the distal end 14 and where the wing-shape is formed as a part of the distal end 140. Utilizing attachments may allow for customization of the tip depending on the physiology and anatomy of a particular patient and may provide a better chance at retrieving a measurable specimen for testing, e.g., for a Pap smear.

By way of a non-limiting example, the distal end 14 may include a generally corkscrew shaped portion 18, as generally illustrated in FIG. 1. The corkscrew portion 18 may be any appropriate size, shape and length, such as approximately 25 mm in length. However, the corkscrew portion 18 may alternatively be longer or shorter than 25 mm, as appropriate. For example, the corkscrew portion may be 1 mm to 50 mm. The shape and size of the corkscrew shaped portion 18 will allow the endocyte cannula 10 to effectively maneuver through the cervix and/or the endocervical canal to effectively obtain the desired cells, especially one at 25 mm.

The corkscrew portion 18 may comprise one or more spirals 20. The spirals 20 may have any appropriate radius. In some embodiments, the radius of the spirals 20 may be approximately 3 mm. The radius of all spirals 20 may be generally consistent, or may vary along the length of the corkscrew portion 18. The shape and size of the spirals 20 will allow the endocyte cannula 10 to effectively maneuver through the cervix and/or the endocervical canal to effectively obtain the desired cells

The distal end 14 or more specifically the corkscrew portion 18 may include a plurality of apertures 22 disposed there along. The apertures 22 may assist the endocyte cannula 10 in advancing through dense tissue in the endocervical canal while at the same time minimizing the risk of perforation to the patient. This design will make the procedure more comfortable for the patient. The apertures 22 may aspirate mucus, thereby minimizing the risk of trauma to the cervical mucosa.

The apertures 22 may be formed through an outer wall of the endocyte cannula 10 and may open into the hollow opening of the endocyte cannula 10, i.e., the tubular portion 13. The apertures 22 may be arranged along the length of the corkscrew portion 18, or along other portions of the endocyte cannula 10. The distal end 14 may include any appropriate number of apertures 22 and are not limited to the number shown in FIGS. 1-6. Further, the apertures 22 may be positioned in any appropriate configuration. By way of a non-limiting example, the apertures may generally be evenly spaced from one another along the corkscrew portion 18. The apertures may generally be unevenly spaced from one another along the corkscrew portion 18. The apertures may be uniform in size or they may vary in size. The apertures may be round in shape, ovoid in shape, rectangular in shape, or any other appropriate shape.

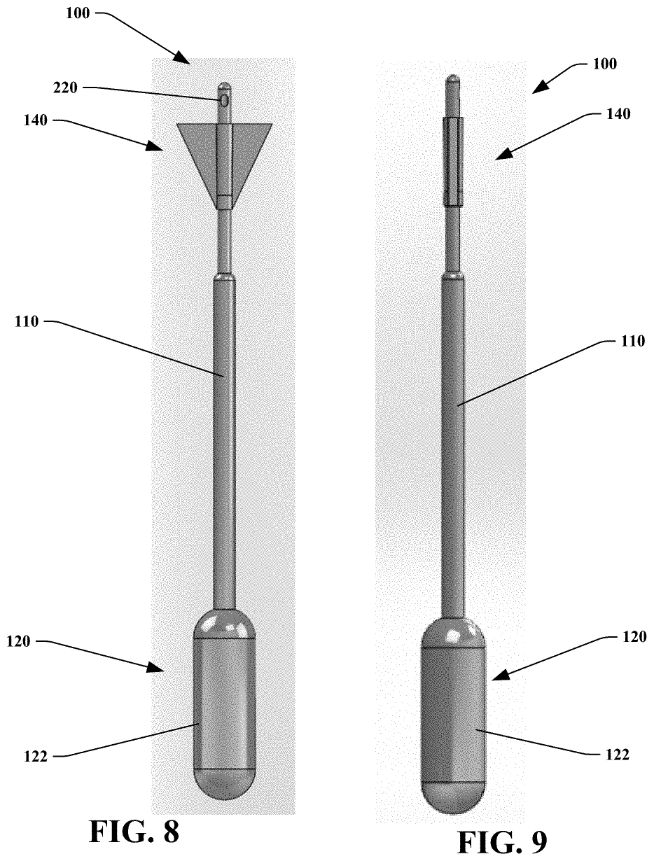

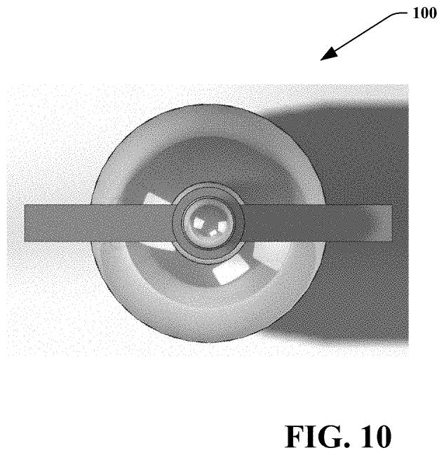

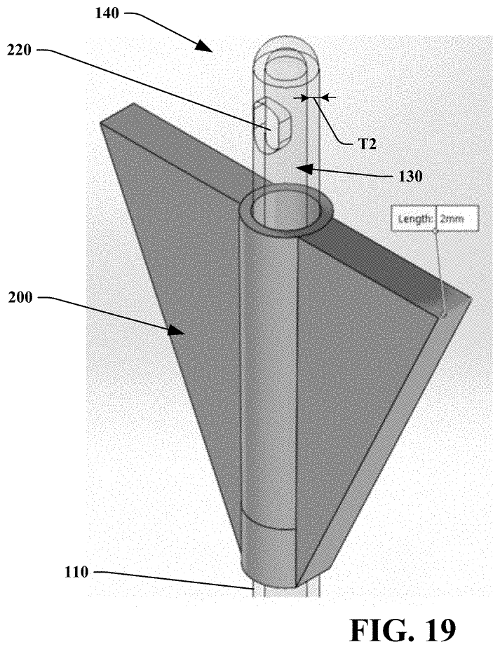

FIG. 8 depicts another embodiment of an endocyte cannula 100. The endocyte cannula 100 may be comprised of any appropriate material, including, without limitation, steel, stainless steel, plastic, rubber, a combination of the foregoing or any other appropriate material. In an embodiment, the endocyte cannula 100 may be comprised of medical grade plastic, may be disposable or single-use, and/or may be made available in translucent colors. The endocyte cannula 100 may include a body 110. The body 110 may be generally hollow and may include any appropriate wall thickness, shown as T2 in FIG. 19. T2 may be any appropriate thickness including, for example, 0.1 mm-10 mm, and also including the same or similar thickness as indicated for endocyte cannula 100 and shown as T in FIG. 2. As shown in FIG. 19, the body 110 may include a generally hollow tubular portion 130, which may be used in the aspiration process described in more detail below. The endocyte cannula 100 may be generally about between 10-25 cm or more specifically about 20 cm (i.e., within 2 to 3 cm of such).

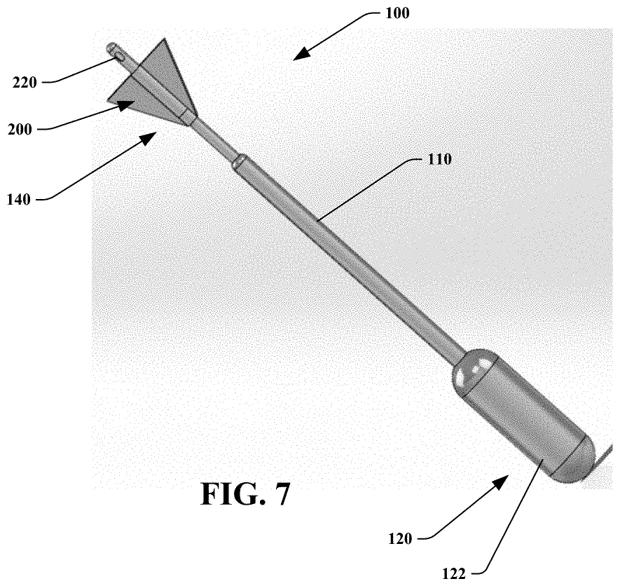

The endocyte cannula 100 may include a proximal end 120 and a distal end 140. The proximal end 120 may be generally configured to connect to a user-control mechanism or may include an aspiration device integrated therein, such as the bulbous end shown in FIG. 7. By way of a non-limiting example, the user control mechanism may include a syringe 15 or the like. By way of another non-limiting example, the user control mechanism may include a bulbous end that when depressed will cause suction at the distal end 140 without the need for a separate device.

The distal end 140 may be designed to generally increase effectiveness of use and allow for aspiration, as further described below. The endocyte cannula 10 and endocyte cannula 100 may generally work the same or similarly insofar as having a hollow tubular body, aspiration, and connection to a user control mechanism of any appropriate embodiment. In other words, any such aspects described for one of the embodiments of the endocyte cannula 10 or endocyte cannula 100 may be applied to the other even if not explicitly described.

The endocyte cannula 100 may be any appropriate size and shape. By way of a non-limiting example, the endocyte cannula 100 may be approximately 12 cm in total length. This length will allow the endocyte cannula 100 to effectively maneuver through the cervix and/or the endocervical canal to effectively obtain the desired cells. It will be appreciated, however, that the length of the endocyte cannula 100 may be longer or shorter than 12 cm, and may be any appropriate length. For example, the endocyte cannula 100 may be 5 cm to 50 cm in length—the length should be configured such that the endocyte cannula 100 is able to effectively maneuver through the cervix and/or the endocervical canal to effectively obtain the desired cells. The diameter of the endocyte cannula 100 may be similar to that of a 20 gauge needle, such as between 0.6 mm and 1.0 mm. Alternatively, the diameter of the endocyte cannula 100 may be greater or smaller than that of a 20 gauge needle, for example, the diameter may range from 0.1 mm to 10 mm. The diameter may be consistent throughout the entirety of the endocyte cannula 100 or may vary across the length or at different portions of the endocyte cannula 100.

The proximal end 120 may be configured to connect to a user control mechanism. For example, the proximal end 120 may include a fitting 16, as similarly described in relation to endocyte cannula 10 and shown in FIGS. 1-6, such as a taper or other feature, to interface with the syringe 15 or the proximal end 120 may include an integrated user control mechanism, such as the bulbous end described in more detail below. In an embodiment, the fitting 16 may comprise a “luer taper” or “luer lock” or any other appropriate type of fitting or means or connection to provide an interface between the endocyte cannula 100 and the syringe 15 or other tool. While the syringe 15 is described as being used with the endocyte cannula 10 it should be understood that any appropriate user control mechanism may be used and the teachings are not limited to the syringe 15 shown and described or the bulbous end described in more detail below.

In some embodiments, the syringe 15 may be selectively attached to the fitting 16, as similarly described in relation to endocyte cannula 10 and shown in FIGS. 4-6. The syringe 15 may interface with the generally hollow endocyte cannula 100, specifically, the tubular portion 130 to apply a force, pressure or suction therethrough. When suction is applied to the cervix, a representative sample of the cells may be collected into the shaft portion or body of the endocyte cannula 100 and then disposed into the designated jar or slide. While the endocyte cannula 100 is described as connected to the syringe 15, it will be appreciated that the proximal end 120 may be connected to any appropriate tool, including any manually actuated, robot actuated or automated tool.

In other embodiments, the proximal end 120 may have a user control mechanism integrated directly therein such that use of a separate device, such as the syringe 15 described above, isn't necessary. By way of a non-limiting example, the proximal end 120 may include a bulbous end 122 that may act as an aspiration device to aspirate the desired cells into the endocyte cannula 100. In this embodiment, the user may depress the bulbous end 122 and upon release suction may be created to operatively move the applicable/desires cells into the endocyte cannula 100 as described herein. While the bulbous end 122 is described and shown, the present teachings aren't limited to this embodiment. The proximal end 120 may include any integrated/monolithically formed user control mechanism that is able to suction/aspirate the relevant cells/mucous into the endocyte cannula 100. Additional embodiments may include a syringe-like end, a plunger or the like. This component may make it unnecessary to use a secondary aspiration device like the syringe 15 described above.

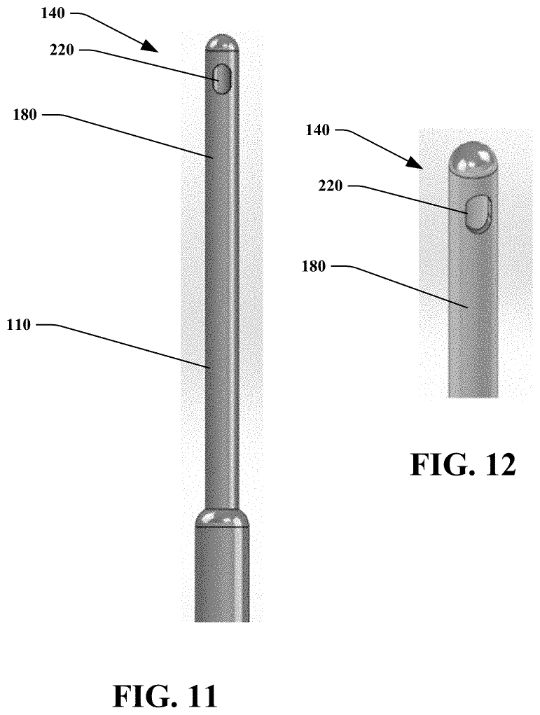

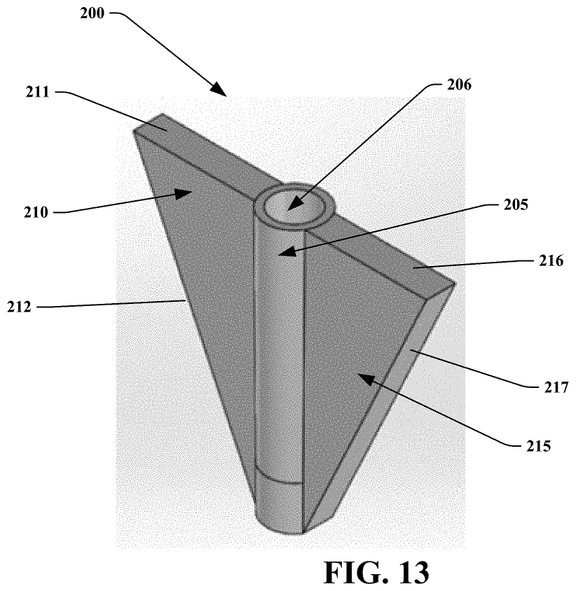

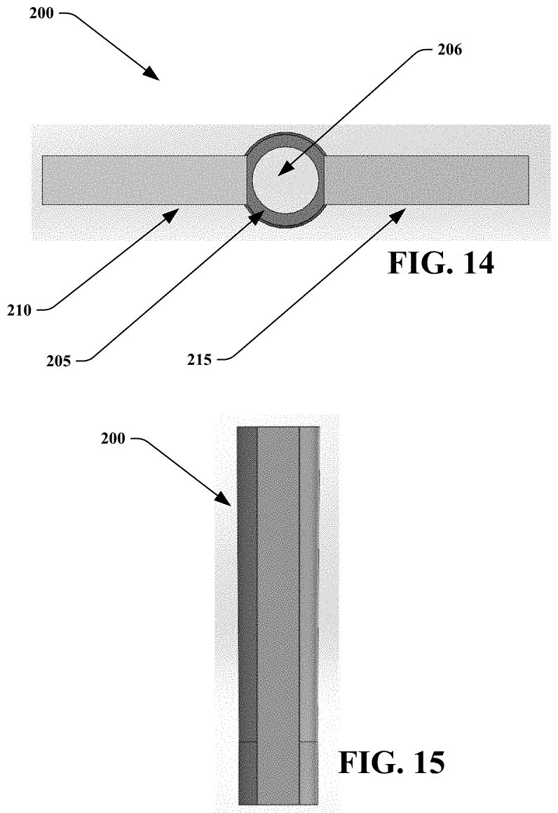

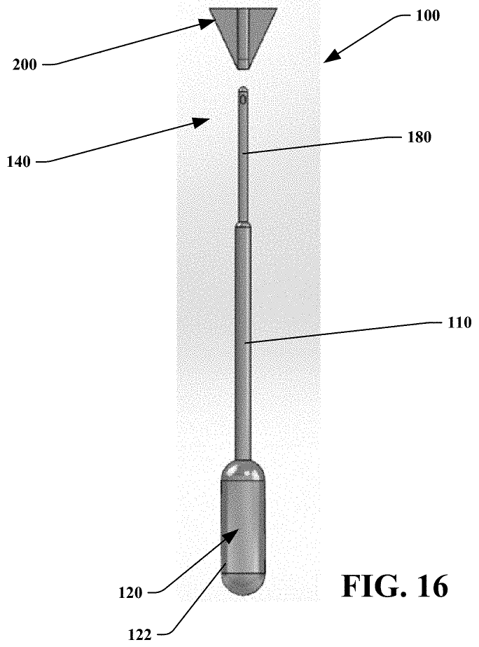

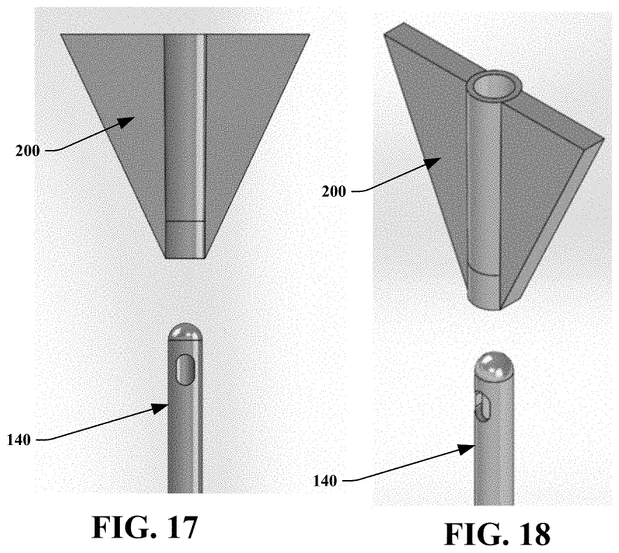

The distal end 140 may be configured to enhance the functionality and use of the endocyte cannula 100, and/or to receive an attachment that assists in these ends. By way of a non-limiting example, the distal end 140 may include a shaft 180 that is configured to receive an attachment 200, as generally illustrated in FIGS. 11-12 and 16-19. Utilizing attachments may allow for customization of the tip depending on the physiology and anatomy of a particular patient and may provide a better chance at retrieving a measurable specimen for testing, e.g., for a Pap smear. In an example, the length and width of the attachment may be selected based on the depth and width of the cervical canal of the patient. The endocyte cannula 100 may have the added benefit of providing a patient with a more comfortable experience. Additionally, the endocyte cannula 100 may minimize the amount of trauma caused by the procedure to the tissue of the cervix, preventing the risk of bleeding (which could obscure the cell component of the Pap smear and compromise the accuracy of the test). Moreover, the endocyte cannula 100 may provide a better yield of mucus or other tissue because it avoids trauma that can contaminate the sample with blood.

The shaft 180 may be any appropriate size, shape and length, such as approximately 25 mm in length in one embodiment and about 4 cm in another embodiment (i.e., within 5 mm of such). However, the shaft 180 may alternatively be longer or shorter than 25 mm, as appropriate. For example, the shaft portion may be 1 mm to 50 mm. The shaft 180 may include at least one aperture 220 as described in more detail below—the at least one aperture 220 may comprise a single aperture. The shaft 180 may have a diameter that is less than the diameter of the body 110 or the shaft 180 and the body 110 may have the same diameter as shown in FIG. 11. The shaft 180 may be rigid, which can help the endocyte cannula 100 navigate its way through the cervix and/or endocervical canal avoiding trauma to the patient.

The shaft 180 may include a stop piece or a locking mechanism to keep the attachment 200 in place on the shaft 180. For example, the stop piece or the locking mechanism may prevent the attachment 200 from being pushed further down the body 110 and/or the stop piece or the locking mechanism may prevent the attachment 200 from unintentionally coming off of the shaft 180 during, for example, use of the endocyte cannula 100 in the cervix and/or endocervical canal. The stop piece or the locking mechanism may include a pressure fit, friction fit, snap fit, sliding mechanism, or other type of fastener to keep the attachment 200 in place on the shaft 180.

The attachment 200 may include a shape configured to provide mechanical scraping, brushing, or otherwise facilitate the positioning of the device during a Pap smear and the collection and retrieval of a desired cellular specimen, including cervical cells (e.g. from the face of the cervix), endocervical cells (e.g. from the endocervical canal), and/or cervical mucous. By way of a non-limiting example, the attachment 200 may include a receiving portion or passage, also referred to as a hollow tubular body, 205 that is configured to receive at least a portion of the shaft 180 there through. In an embodiment, the shaft 180 may extend entirely through the hollow tubular body 205 of the attachment 200 and through an opposite opening 206 of the hollow tubular body 205. In an embodiment, the shaft 180 may extend entirely through the hollow tubular body 205 and the attachment 200, through an opposite opening 210 of the hollow tubular body 205 and the at least one aperture 220 may be positioned on the portion of the shaft 180 that is so extended, see FIGS. 7-8 and 19.

The attachment 200 may further include at least one wing 215, extending laterally from the hollow tubular body 205. In an embodiment, the attachment 200 may further include at least two wings 210, 215 extending laterally from the hollow tubular body 205 (although three, four, five, six or more wings may be utilized). In an embodiment, the at least two wings 210, 215 may extend opposite the hollow tubular body 205. In an embodiment, the at least one wing 210 may have a generally triangular shape. In an embodiment, the at least one wing 210 may have two edges 211, 212 of different lengths. In an embodiment, the two edges 211, 212 of the at least one wing may have the same length. In an embodiment, the second wing 215 may have a generally triangular shape. In an embodiment, the second one wing 215 may have two edges 216, 217 of different lengths. In an embodiment, the two edges 216, 217 of the second wing may have the same length. In an embodiment, the lateral or perpendicular width of the attachment 200, e.g. two edges 211, 216, may be 15-25 mm. In an embodiment, the length of the attachment 200, e.g., the hollow tubular body 205, may be 13-23 mm. In an embodiment, the thickness of the wings 210, 215 may be 1-3 mm and the exterior diameter of the hollow tubular body 205 may be 2.95-4.95 mm and the interior diameter of the hollow tubular body 205 may be 1.75-3.75 mm. It is noted that these measurements are exemplary and that the actual length, width, thickness, and diameters of the attachment 200 may fall outside of these ranges. The size and configuration of the wings 210, 215 may allow the endocyte cannula 100 to be operatively positioned into and through the cervix and/or endocervical canal to collect mucous or other cells in a manner that significantly reduces or eliminates trauma to the patient. This in turn may provide a better yield of mucous and/or cells by reducing or eliminating contamination from blood caused by trauma.

In an embodiment, the first wing 210 and the second wing 215 may match or be a mirror image of one another. For example, one corresponding edge 211, 216 of the first wing 210 and the second wing 215 may generally be the same length and angle from the hollow tubular body 205. A second corresponding edge 212, 217 of the first wing 210 and the second wing 215 may generally be the same length and angle from the hollow tubular body 205. Although a generally triangular shape of the at least one wing 210 and the second wing 215 may be generally described and shown in the drawings, it is noted that any suitable shape may be used and any shape having any number and lengths of edges, including 1, 2, 3, 4, 5, 6, 7, or more edges. The shape of the at least one wing 210 and the second wing 215 may be round, triangular, square, irregular, asymmetrical, etc. The shape of the at least one wing 210 and the second wing 215 may be the same, similar, or different from one another. Utilizing attachments may allow for customization of the tip depending on the physiology and anatomy of a particular patient and may provide a better chance at retrieving a measurable specimen for testing, e.g., for a Pap smear. In an example, the length and width of the attachment may be selected based on the depth and width of the cervical canal of the patient. The endocyte cannula 100 may have the added benefit of providing a patient with a more comfortable experience. Additionally, the endocyte cannula 100 may minimize the amount of trauma caused by the procedure to the tissue of the cervix, preventing the risk of bleeding (which could obscure the cell component of the Pap smear and compromise the accuracy of the test).

The distal end 140, the shaft 180, and/or the attachment 200 may include one or more apertures 220 disposed there along. In an embodiment, the aperture 220 on the attachment 200 may be fluidly related to the hollow body 110. The aperture 220 may assist the endocyte cannula 100 in advancing through dense tissue in the endocervical canal while at the same time minimizing the risk of perforation to the patient. The aperture 220 may aspirate mucus, thereby minimizing the risk of trauma to the cervical mucosa.

The aperture 220 may be formed through an outer wall of the endocyte cannula 100 and may open into the hollow opening of the endocyte cannula 100, i.e., the tubular portion 130 as shown in FIG. 19. The aperture 220 may be arranged along the distal end 140, or along other portions of the endocyte cannula 100. While one aperture 220 is shown and described, the distal end 140 may include any appropriate number of apertures 220 (e.g., 2, 3, 4, 5, 6, etc.) and are not limited to the number shown in any of the Figures. Further, the one or more apertures 220 may be positioned in any appropriate configuration. By way of a non-limiting example, the apertures 220 may generally be evenly spaced from one another along the distal end 140. The apertures 220 may generally be unevenly spaced from one another along the distal end 140. The apertures 220 may be uniform in size or they may vary in size. The one or more apertures 220 may be round in shape, ovoid in shape, rectangular in shape, or any other appropriate shape.

In an embodiment, the shaft 180 of the endocyte cannula 100 extends entirely through the hollow tubular body 205 of the attachment 200 and through an opposite opening 206 of the hollow tubular body 205 of the attachment 200. In this embodiment, the one or more apertures 220 are positioned on the distal end 140 and specifically on the portion of the distal end 140 or shaft 180 that extended through the opposite opening 206 of the hollow tubular body 205 of the attachment 200 as shown in FIG. 19. In this embodiment, the attachment 220 comprises two triangular wings 210, 215 that have lateral edges 211, 216 extending perpendicularly from the hollow tubular body 205 of the attachment 200 or the distal end 140 or shaft 180, and angular edges 212, 217 that extend from the lateral edges 211, 216 and to the hollow tubular body 205 of the attachment 200.

In this and other embodiments described herein, the narrow distal end 140 that extends through the attachment 200 may more easily slide inside the endocervical canal and, having one or more apertures 220 thereon, may facilitate the aspiration of the endocervical mucous inside the endocyte cannula 100. In other words, the endocyte cannula 100, having a narrow and exposed distal tip 140 and/or lateral wing attachment 220 may allow for easier penetration into the endocervical canal. The endocyte cannula 100 may streamline the process of collecting a sample by combining both a brush (scraping) and spatula/aspiration (retrieving) aspects, and providing a hybrid endocyte cannula 100. The collection process may be faster, may occur in one step instead of two, and save costs. The hybrid endocyte cannula 100, having both scraping and retrieving abilities, may produce a better yield and a more representative sample from the cervix while minimizing bleeding and a patient's discomfort during the procedure. The same or similar benefits may also be found in respect to the endocyte cannula 10, having a corkscrew portion. Additionally, utilizing attachments may allow for customization of the tip depending on the physiology and anatomy of a particular patient and may provide a better chance at retrieving a measurable specimen for testing, e.g., for a Pap smear. In an example, the length and width of the attachment may be selected based on the depth and width of the cervical canal of the patient.

The endocyte cannula 10, 100 may also include other features, for example, bristles, rough surfaces, smooth surfaces, reflective surfaces, or matte surfaces. The endocyte cannula 10, 100 may also include a light, a camera, or any other feature beneficial to the operator. Further still, each of the components of the endocyte cannula 10 may be utilized with or added onto the endocyte cannula 100 and each of the components of the endocyte cannula 100 may be utilized with or added onto the endocyte cannula 10,

Although the embodiments of the present invention have been illustrated in the accompanying drawings and described in the foregoing detailed description, it is to be understood that the present invention is not to be limited to just the embodiments disclosed, but that the invention described herein is capable of numerous rearrangements, modifications and substitutions without departing from the scope of the claims hereafter. The claims as follows are intended to include all modifications and alterations insofar as they come within the scope of the claims or the equivalent thereof.

1. A medical device comprising:
a tubular body having a proximal end and a distal end, wherein the tubular body is rigid and includes a hollow channel, wherein the distal end is configured to receive a scraping attachment, and wherein the proximal end includes a user-control mechanism.

2. The medical device of claim 1, wherein the scraping attachment is triangular.

3. The medical device of claim 1, wherein the scraping attachment includes two lateral wings.

4. The medical device of claim 1, wherein the scraping attachment is corkscrew shaped.

5. The medical device of claim 1, wherein the distal end further comprises at least one aperture, the aperture having a first opening through an outer wall of the distal end and a second opening through an inner wall of the hollow channel of the tubular body.

6. The medical device of claim 1, wherein the scraping attachment includes at least one aperture, the aperture having a first opening through an outer wall of the scraping attachment and a second opening through an inner wall a hollow channel of the scraping attachment, wherein the hollow channel of the scraping attachment is fluidly connected to the hollow channel of the tubular body when the scraping attachment is attached to the tubular body.

7. The medical device of claim 1, wherein the scraping attachment includes a receiving passage configured to receive the distal end of the tubular body there through.

8. The medical device of claim 7, wherein the receiving passage extends through an entire length of the scraping attachment, having a first opening on a first end and a second opening on a second end, and wherein the distal end of the tubular body extends into the first opening, through the length of the scraping attachment, and at least a potion of the distal end of the tubular body extends out of the second opening, when the scraping attachment is attached to the tubular body.

9. The medical device of claim 8, wherein at least one aperture is located on the portion of the distal end that extends through the scraping attachment and second opening when the scraping attachment is attached to the tubular body.

10. The medical device of claim 1, wherein the user-control mechanism is a syringe.

11. The medical device of claim 1, wherein the user-control mechanism is a bulbous end.

12. The medical device of claim 1, wherein the user-control mechanism interfaces with the hollow tubular body to create a force through the medical device.

13. The medical device of claim 1, wherein the medical device is configured to aspirate bodily tissues.

14. The medical device of claim 1, wherein the distal end and scraping attachment are configured to be received into a body cavity.

15. The medical device of claim 14, wherein the scraping attachment is configured to collect cells from the body cavity.

16. The medical device of claim 15, wherein the body cavity is an endocervical canal.

17. The medical device of claim 1, wherein the hollow tubular body comprises steel, stainless steel, plastic, rubber, or a combination of two or more thereof.

18. A method for completing a Papanicolaou test comprising:
inserting a medical device into an endocervical canal,
the medical device comprising a hollow tubular body having a proximal end and a distal end, wherein the proximal end connects to a user-control mechanism and the distal end comprises a scraping component and at least one aperture; and
collecting cells from the endocervical canal with the medical device.

19. The method of claim 18, wherein the distal end and scraping component advance through bodily tissue.

20. The method of claim 19, wherein the at least one aperture aspirates bodily tissues.
